(19) United States
(12) Patent Application Publication
(10) Pub. No.: US 2022/0372429 A1
(43) Pub. Date: Nov. 24, 2022

(54) ALGAE CULTIVATION SYSTEMS AND METHODS WITH HIGH LIPID AND PROTEIN CONTENT

(71) Applicant: Global Algae Technology, LLC, Santee, CA (US)

(72) Inventors: Agnieszka Pinowska, San Diego, CA (US); Rodney Corpuz, Lihue, HI (US); David A. Hazlebeck, El Cajon, CA (US)

(21) Appl. No.: 17/746,448

(22) Filed: May 17, 2022

Related U.S. Application Data
(60) Provisional application No. 63/190,678, filed on May 19, 2021.

(51) Int. Cl. C12N 1/12; C12N 1/02; C12N 1/38

(52) U.S. Cl. CPC C12N 1/12; C12N 1/02; C12N 1/38; C12R 2001/89

(57) ABSTRACT

A method of autotrophic cultivation of algae includes cultivating algae in the presence of cultivation media and at least one growth nutrient to produce an algal biomass; and harvesting the algal biomass by separating the algal biomass from the cultivation media and the at least one growth nutrient. The algal biomass comprises at least 35% fatty acid lipid content and at least 30% protein content on an ash-free dry weight basis

### CROSS-REFERENCE TO RELATED APPLICATIONS

The present application claims the benefit of U.S. Provisional Patent Application No. 63/190,678 that was filed May 19, 2021, the entire contents of which are hereby incorporated by reference.

### STATEMENT REGARDING FEDERALLY SPONSORED RESEARCH

This invention was made with government support under award #DE-EE0008516 and award #DE-EE0008903, both awarded by the Department of Energy (“DOE”). The government has certain rights in the invention.

### FIELD OF THE DISCLOSURE

The present disclosure relates generally to algae cultivation systems and methods, and more particularly to algae cultivation systems and methods that provide a combination of high fatty acid content with high protein content resulting in greater product value.

### BACKGROUND OF THE DISCLOSURE

Algae cultivation has become widely recognized as a promising source of food, biofuel, chemicals, and nutraceuticals. The major constituents of most microalgae are fatty acid lipids, proteins, and carbohydrates. Some species microalgae, such as Botryococcus braunii, also have hydrocarbon lipids as a major component. Of these three or four major constituents, the fatty acid lipids and proteins have the highest value, so obtaining algae that is high in both constituents is beneficial.

Unfortunately, for autotrophic algae cultivation, algae with higher fatty acid lipid content have lower protein content. For example, high fatty acid lipid content algae, e.g. at least 35%, does not have at least 30% protein content. It should be appreciated that achieving at least 35% fatty acid lipid content with at least 30% protein content would improve the economics for large-scale algae cultivation. There are multiple benefits to producing both fatty acid based algal oil and high protein content algal meal. Firstly, the ratio of protein to fatty acids can be adjusted based on the immediate market conditions to maximize the value of the biomass. Secondly, protein productivity of algae is much greater than for terrestrial crops for a given land or water input; therefore, even, if a significant portion of the algal oil is going to biofuels, there is no fuel vs food trade-off because more food is generated from the co-product protein meal than could have been generated with the same land and water inputs using terrestrial crops and no biofuel production. As the fatty acid lipid content is increased above 40%, the target protein content can be reduced as long as the combined lipid and protein content is at least 70% without sacrificing the economics; thus, it should also be appreciated that achieving at least 40% fatty acid lipid content and a combined protein and fatty acid lipid content of at least 70% would improve the economics for large-scale algae cultivation.

### BRIEF SUMMARY OF THE DISCLOSURE

The present disclosure provides algae cultivation systems and methods that can produce algae with at least 35% fatty acid lipid content in combination with at least 30% protein content on an ash-free dry weight basis. The disclosure further provides algae cultivation systems and methods that can produce algae with greater than 40% fatty acid lipid content in combination with greater than 30% protein content on an ash-free dry weight basis. The methods include two-stage cultivation. In a growth stage, algae is cultivated autotrophically under nutrient replete conditions to obtain a high productivity and high protein content. Then in a lipid stage, the algae are cultivated with at least one limiting nutrient other than nitrogen for a period of up to 7 days. The nutrient limitation causes fatty acid accumulation in the cell. The protein content does not decrease precipitously because sufficient nitrogen is maintained. The protein decrease over time is reduced because the time for nutrient limitation is 7 days or shorter, and preferably 5 days or shorter, more preferably 3 days or shorter, and most preferably 2 days or shorter.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 is a schematic illustration of an exemplary algae system for performing two-stage cultivation according to some embodiments of the present invention; and

FIG. 2 is an exemplary graph illustrating the range of fatty acid lipid content and protein content attained previously (open circles) compared to target range and the content achieved with some embodiments of the present disclosure (closed circles).

### DETAILED DESCRIPTION

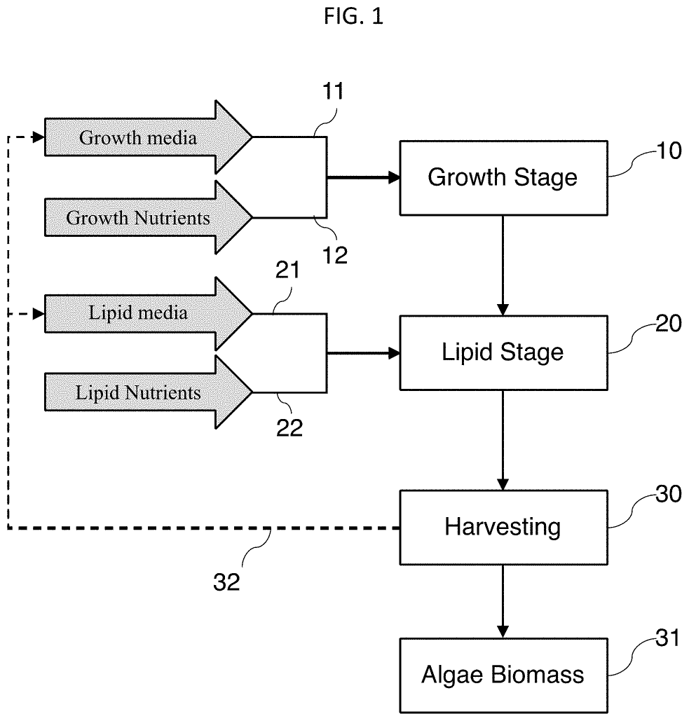

Referring now to FIG. 1, a system for autotrophically growing algae is illustrated with a first growth stage 10 and a second lipid stage 20. In some embodiments, the first growth stage 10 contains algae in cultivation media. The first growth stage 10 may be placed in fluid communication with a growth media source 11 that supplies cultivation or growth media to the growth stage 10. The first growth stage 10 may be further placed in fluid communication with a growth nutrient source 12 that supplies at least one nutrient to the growth stage 10. In some embodiments, the growth media may be added to the first growth stage 10 to make up for evaporation and to maintain the desired concentration of algae as the algae grows. In some embodiments, the at least one nutrient may be added to provide replete nutrients for growth of the algae.

At least a portion of the alga in cultivation media from the growth stage 10 is moved to the lipid stage, 20. The lipid stage 20 may be placed in fluid communication with a lipid media source 21 that provides lipid media to the lipid stage 20. The lipid stage 20 may be further placed in fluid communication with a lipid nutrient source 22 that provides at least one nutrient to the lipid stage 20. In some embodiments, the lipid media is added to make up for evaporation and to maintain the desired concentration of algae as the algae grows in the lipid stage 20. In some embodiments, the at least one nutrient from the lipid nutrient source 22 contains sufficient nitrogen for the algae growth, but at least one nutrient required for growth is limited.

Exemplary nutrients that may be limited include, but are not limited to, fertilizing nutrients (e.g., nitrogen, phosphorous, potassium, or combinations thereof), other macronutrients (e.g. silica, calcium, magnesium, sodium, chlorine, sulfate), and (e.g., iron, boron, borate, manganese, molybdenum, zinc, copper, iodine, bromine, tungstate, chromium, cadmium, nickel, aluminum, vanadium). In some embodiments, the limited nutrient (e.g., silica) in the lipid stage 20 is present at a concentration of 1 to 8 millimolar (mM)/g dry weight algae. In some embodiments, the limited nutrient is present in the lipid stage 20 at a concentration (including silica already incorporated into the algae) of less than 8 mM/g dry weight algae, or less than 7 mM/g dry weight algae, or less than 6 mM/g dry weight algae, or less than 5 mM/g dry weight algae.

As the algae in the lipid stage 20 grows, the at least one limiting nutrient becomes exhausted or nearly exhausted from the media surrounding the algae. In response, the algae accumulate fatty acid lipids. After a period of time (e.g., 7-days or less, 5-days or less, 3-days or less, 2-days or less, or 1-days or less) after the limiting nutrient becomes exhausted or nearly exhausted, the algae in the lipid stage 20 is transferred to a harvesting stage 30 to produce an algae biomass 31 and substantially algae-free cultivation media 32, where at least one limiting nutrient is exhausted or nearly exhausted in the algae-free cultivation media 32. The cultivation media 32 is optionally recycled to the growth stage 10 or the lipid stage 20. The algae biomass 31 has at least 35% fatty acid lipid content and at least 30% protein content on an ash-free dry weight basis, or has at least 40% fatty acid lipid content and a total fatty acid lipid content plus protein content of at least 70%.

In some embodiments, the algae biomass 31 has a fatty acid lipid content of at least 35% on a ash-free dry weight basis, or at least 36%, or at least 37%, or at least 38%, or at least 39%, or at least 40%, or at least 45%, or at least 50%, or at least 55%, or at least 60% on a ash-free dry weight basis, and any range between the specified values (e.g., from 35% to 60%, from 35% to 55%, from 37% to 50%, etc.). In some embodiments, the algae biomass 31 has a protein content of at least 20% on a dry ash-free dry weight basis, or at least 25%, or at least 30%, or at least 31%, or at least 32%, or at least 33%, or at least 34%, or at least 35%, or at least 36%, or at least 37%, or at least 38%, or at least 39%, or at least 40% on a dry ash-free weight basis, and any range between the specified values (e.g., from 30% to 40%, from 30% to 35%, from 35% to 38%, etc.). In some embodiments, the total amount of fatty acid lipid and protein is at least 60%, or at least 65%, or at least 70%, or at least 75%, or at least 80% on a dry ash-free weight basis, and any range between the specified values (e.g., from 60% to 80%, from 70% to 80%, etc.).

During autotrophic growth, algae fix carbon through photosynthesis. Chlorophyll is a key component for photosynthesis, so under nutrient replete conditions, the chlorophyll content is typically at least 1% of the ash-free dry weight. During lipid formation the chlorophyll content can be decreased by up to 10-fold, so the chlorophyll content can be as low as 0.1% of the ash-free dry weight. In some embodiments, the algae biomass 31 contains chlorophyll at a concentration of at least 0.1% of the ash-free dry weight, or at least 0.2%, or at least 0.3%, or at least 0.4%, or at least 0.5%, or at least 1% of the ash-free dry weight of the algae biomass 31.

It will be appreciated by one skilled in the art of algae cultivation that the growth phase and lipid phase can be performed in the same open pond bioreactor or closed photobioreactor or in separate open pond bioreactors or closed photobioreactors. Also, the nutrients can be added directly to the cultivation system or to the media, and the media may be fresh or brackish water and may contain sodium carbonated or bicarbonate. Furthermore, the algae may be partially harvested in moving from the growth phase to the lipid phase to increase the concentration, and the amount of media addition may be adjusted to obtain a more concentrated or more dilute algae slurry in the lipid phase. The lipid media and growth media can also be distinct media with separate harvesting steps as described U.S. Pat. No. 10,501,721.

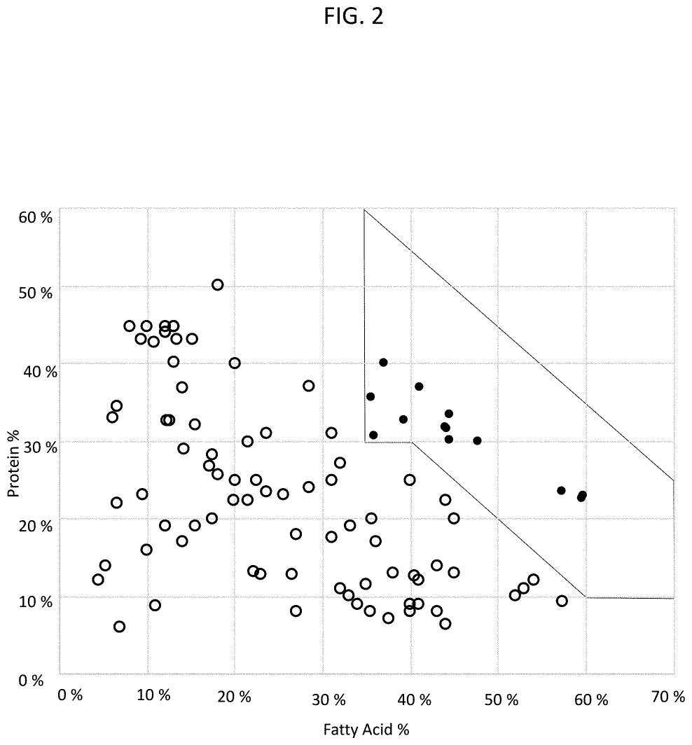

Referring now to FIG. 2, the open circles are literature values for algae fatty acid lipid content and protein content from sources: Ajjawi et al. 2017; Amorim et al. 2021; Ansari et al 2015; Blifernez-Klassen et al. 2018; Breuer et al. 2012; Cheng et al 2014; Chokshi et al. 2017; Chu et al. 2014; Duong et al. 2015; Eustance et al. 2015; Ho et al. 2011; Laurens et al 2014; Laurens et al 2017a; and Laurens et al 2017b. Fuller citations to these sources are provided below. The shaded area represents the target fatty acid lipid content and protein content for improve large-scale algae economics. The solid circles are algae biomass content achieved according to some embodiments of the present disclosure.

In addition to increasing the product value, the economics of large-scale algae cultivation can be improved by lowering the cost of production. Autotrophic growth in open raceways is one of the least expensive systems for large-scale production of algae. Algae requires carbon dioxide for autotrophic growth. Typically carbon dioxide or bicarbonate is added to the cultivation media to support growth of the algae. During cultivation, if the pH is less than about 9, then a significant amount of the added carbon dioxide is lost from the media to the atmosphere during cultivation, Thus, operation at a pH of greater than 9, and preferably greater than 9.4 is desirable to decrease the cost of production. Carbon dioxide can also be captured directly from the atmosphere. The pH should preferably be greater than about 9.7 and preferably greater than 10 to capture carbon dioxide directly from the atmosphere.

### Definitions

As used herein, the term “ash-free dry weight” refers to an organic content of the algae determined by rinsing the external dissolved solids and ashing the algae in a furnace at least 500° C. for at least 2 hours.

As used herein, the term “fatty acid lipid content” refers to the total fatty acid content determined by esterification and gas chromatography/mass spectrometry divided by the ash-free dry weight of the sample.

As used herein, the term “protein content” refers to the total protein divided by the ash free dry weight where the protein is determined by amino acid quantification or the nitrogen content measured in a CHN analyzer times the ratio of protein to nitrogen determined for the specific algae species and growth conditions, typically 4.5 to 5.2 for microalgae.

As used herein, the term “open pond bioreactor” refers to a raceway or pond that is open to the atmosphere containing algae in a cultivation media that typically includes a method for mixing algae and media.

As used herein, the term “photobioreactor” refers to a closed cultivation system that contains algae in a cultivation media and a means of removing oxygen from the system.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

Various example embodiments, examples and/or simulations of the systems and methods of the present disclosure are discussed below.

### Example 1

Nitzschia inconspicua was cultivated autotrophically in an open pond bioreactor with replete media at a pH of greater than 9.4 in growth phase. Then, the nutrient addition was changed to a formulation with no silica, i.e., silica was the limiting nutrient. The algae biomass was harvested seven days after changing to silica limited nutrient addition. The biomass was analyzed for fatty acid lipid content and protein content. The fatty acid lipid content was 36% of the ash-free dry weight, and the protein content was 31% of the ash-free dry weight.

### Example 2

Nitzschia inconspicua was cultivated autotrophically with replete media in growth phase. Then, the nutrient addition was changed to a formulation with no silica, i.e., silica was the limiting nutrient. Two days after changing to silica limited nutrient addition, the algae biomass was harvested. The biomass was analyzed for fatty acid lipid content and protein content. The fatty acid lipid content was 37% of the ash-free dry weight, and the protein content was 40% of the ash-free dry weight.

### Example 3

Nitzschia inconspicua was cultivated autotrophically with replete media in growth phase. Then, the nutrient addition was changed to a formulation with no silica, i.e., silica was the limiting nutrient. Four days after changing to silica limited nutrient addition, the algae biomass was harvested. The biomass was analyzed for fatty acid lipid content and protein content. The fatty acid lipid content was 48% of the ash-free dry weight, and the protein content was 30% of the ash-free dry weight.

### Example 4

Nitzschia inconspicua was cultivated autotrophically with replete media in growth phase. Then, the nutrient addition was changed to a formulation with no silica, i.e., silica was the limiting nutrient. Four days after changing to silica limited nutrient addition, the algae biomass was harvested. The biomass was analyzed for fatty acid lipid content and protein content. The fatty acid lipid content was 60% of the ash-free dry weight, and the protein content was 23% of the ash-free dry weight.

### Example 5

Nitzschia inconspicua was cultivated autotrophically with replete media in growth phase. Then, the nutrient addition was changed to a formulation with no silica, i.e., silica was the limiting nutrient. Four days after changing to silica limited nutrient addition, the algae biomass was harvested. The biomass was analyzed for fatty acid lipid content and protein content. The fatty acid lipid content was 41% of the ash-free dry weight, and the protein content was 37% of the ash-free dry weight.

### REFERENCES

Ajjawi et al. 2017. Lipid production in Nannochloropsis gaditana is doubled by decreasing expression of a single transcriptional regulator. Nature Biotechnology 35(7), p 647-652
Amorim et al. 2021. Pilot-scale biorefining of Scenedesmus obliquus for the production of lipids and proteins. Separation and Purification Technology 270 (2021) 118775. https://doi.org/10.1016/j.seppur.2021.118775
Ansari et al 2015. Lipid extracted algae as a source for protein and reduced sugar: A step closer to the biorefinery. Bioresource Technology 179 (2015) 559-564. http://dx.doi.org/10.1016/j.biortech.2014.12.047
Arora et al 2016. Synergistic dynamics of nitrogen and phosphorous influences lipid productivity in Chlorella minutissima for biodiesel production. Bioresource Technology 213 (2016) 79-87. http://dx.doi.org/10.1016/j.biortech.2016.02.112
Blifernez-Klassen et al. 2018. Metabolic survey of Botryococcus braunii: Impact of the physiological state on product formation. PLoS ONE 13(6): e0198976. https://doi.org/10.1371/journal.pone.0198976
Breuer et al. 2012. The impact of nitrogen starvation on the dynamics of triacylglycerol accumulation in nine microalgae strains. Bioresource Technology 124 (2012) 217-226. http://dx.doi.org/10.1016/j.biortech.2012.08.003
Cheng et al 2014. Enhancing the lipid content of the diatom Nitzschia sp. by 60Co-g irradiation mutation and high-salinity domestication. Energy 78 (2014) 9-15. http://dx.doi.org/10.1016/j.energy.2014.06.009
Chokshi et al. 2017. Salinity induced oxidative stress alters the physiological responses and improves the biofuel potential of green microalgae Acutodesmus dimorphus. Bioresource Technology 244 (2017) 1376-1383. http://dx.doi.org/10.1016/j.biortech.2017.05.003
Chu et al. 2014. Effect of phosphorus on biodiesel production from Scenedesmus obliquus under nitrogen-deficiency stress. Bioresource Technology 152 (2014) 241-246. http://dx.doi.org/10.1016/j.biortech.2013.11.013
Duong et al. 2015. High protein- and high lipid-producing microalgae from northern Australia as potential feedstock for animal feed and biodiesel. Frontiers in Bioengineering and Biotechnology, 18 May 2015. https://doi.org/10.3389/fbioe.2015.00053
Eustance et al. 2015. The effects of cultivation depth, areal density, and nutrient level on lipid accumulation of Scenedesmus acutus in outdoor raceway ponds. J Appl Phycol published online: 10 Sep. 2015. DOI 10.1007/s10811-015-0709-z
Ho et al. 2011. Effect of light intensity and nitrogen starvation on CO2 fixation and lipid/carbohydrate production of an indigenous microalga Scenedesmus obliquus CNW-N. Bioresource Technology 113 (2012) 244-252. doi:10.1016/j.biortech.2011.11.133
Laurens et al 2014. Strain, biochemistry, and cultivation-dependent measurement variability of algal biomass composition. Analytical Biochemistry 452 (2014) 86-95. http://dx.doi.org/10.1016/j.ab.2014.02.009
Laurens et al 2017a. Development of algae biorefinery concepts for biofuels and bioproducts; a perspective on process-compatible products and their impact on cost-reduction. Energy Environ. Sci., 2017, 10, 1716. DOI: 10.1039/c7ee01306j
Laurens et al 2017b. Harmonization of experimental approach and data collection to streamline analysis of biomass composition from algae in an inter-laboratory setting. Algal Research 25 (2017) 549-557. http://dx.doi.org/10.1016/j.algal.2017.03.029

1. A method of autotrophic cultivation of algae, the method comprising:
cultivating algae in the presence of cultivation media and at least one growth nutrient to produce an algal biomass; and
harvesting the algal biomass by separating the algal biomass from the cultivation media and the at least one growth nutrient, and wherein the algal biomass comprises at least 35% fatty acid lipid content and at least 30% protein content on an ash-free dry weight basis.

2. The method of claim 1 further comprises limiting at least one nutrient for algae growth other than nitrogen such that the nutrient becomes exhausted or nearly exhausted in the cultivation media.

3. The method of claim 2 further comprising cultivating said algae for up to 7 days after said at least one nutrient is exhausted or nearly exhausted.

4. The method of claim 2 further comprising cultivating said algae for up to 5 days after said at least one nutrient is exhausted or nearly exhausted.

5. The method of claim 2 further comprising cultivating said algae for up to 3 days after said at least one nutrient is exhausted or nearly exhausted.

6. The method of claim 2 wherein said autotrophic cultivation includes a growth phase prior to said limiting at least one nutrient, and said cultivation media is replete in the nutrients required for algae growth during said growth phase.

7. The method of claim 1 wherein said fatty acid lipid content is at least 40%.

8. The method of claim 1 wherein said fatty acid lipid content is at least 45%.

9. The method of claim 1 wherein said protein content is at least 35%.

10. The method of claim 1 wherein said algae is a diatom.

11. The method of claim 10 wherein said diatom is from the genus Nitzschia.

12. A method of autotrophic cultivation of algae, the method comprising:
cultivating algae in the presence of cultivation media and at least one growth nutrient to produce an algal biomass; and
harvesting the algal biomass by separating the algal biomass from the cultivation media and the at least one growth nutrient, wherein the algal biomass comprises at least 40% fatty acid lipid content on an ash-free dry weight basis and a total of the fatty acid lipid content plus a protein content of at least 70% on an ash-free dry weight basis.

13. The method of claim 12 further comprising limiting at least one nutrient for algae growth other than nitrogen such that the nutrient becomes exhausted or nearly exhausted in the cultivation media.

14. The method of claim 13 further comprising cultivating said algae for up to 7 days after said at least one nutrient is exhausted or nearly exhausted.

15. The method of claim 13 further comprising cultivating said algae for up to 5 days after said at least one nutrient is exhausted or nearly exhausted.

16. The method of claim 13 further comprising cultivating said algae for up to 3 days after said at least one nutrient is exhausted or nearly exhausted.

17. The method of claim 13 wherein said autotrophic cultivation includes a growth phase prior to said limiting at least one nutrient, and said cultivation media is replete in the nutrients required for algae growth during said growth phase.

18. The method of claim 12 wherein said total of the fatty acid lipid content plus the protein content is at least 80%.

19. The method of claim 12 wherein said fatty acid lipid content is at least 50%.

20. The method of claim 12 wherein said algae is a diatom.

21. The method of claim 20 wherein said diatom is from the genus Nitzschia.

22. An algal biomass comprising: (i) at least 35% fatty acid lipid content, (ii) at least 30% protein content, and (iii) at least 0.1% chlorophyll, each on an ash-free dry weight basis.

23. The algal biomass of claim 22 comprising from 35% to 60% fatty acid lipid content on an ash-free dry weight basis.

24. The algal biomass of claim 22 comprising from 30% to 40% protein content on an ash-free dry weight basis.

25. The algal biomass of claim 22 comprising from 0.1% to 1% chlorophyll on an ash-free dry weight basis.

26. The algal biomass of claim 22, wherein a total content of the fatty acid lipid and protein content is at least 70% on an ash-free dry weight basis.
